(19) United States
(12) Patent Application Publication
(10) Pub. No.: US 2016/0095920 A1
(43) Pub. Date: Apr. 7, 2016

(54) KRAS MUTATIONS AND RESISTANCE TO ANTI-EGFR TREATMENT

(71) Applicant: Memorial Sloan- Kettering Cancer Institute, New York, NY (US)

(72) Inventors: Alberto Bardelli, Turin (IT); Federica Di Nicolantonio, La Loggia (IT); Salvatore Siena, Milan (IT); David B. Solit, New York, NY (US)

(21) Appl. No.: 14/790,492

(22) Filed: Jul. 2, 2015

Related U.S. Application Data
(63) Continuation of application No. 13/899,562, filed on May 21, 2013, now abandoned.
(60) Provisional application No. 61/650,253, filed on May 22, 2012.

(51) Int. Cl. A61K 39/395; G01N 33/574; A61K 31/519; C12Q 1/68; A61K 31/4427; A61K 31/21

(52) U.S. Cl. CPC A61K 39/3955; A61K 31/4427; A61K 31/21; A61K 31/519; C12Q 1/6886; G01N 33/57419; C12Q 2600/106; C12Q 2600/158; C12Q 2600/156; G01N 2333/914; G01N 2800/52

(57) ABSTRACT

The disclosure provides compositions and methods for detecting and predicting acquired resistance to anti-EGFR treatment in colorectal cancers. Also provided are compositions and methods of preventing, reversing or delaying the acquired resistance. The present disclosure also provides kits for use in the methods described herein.

### RELATED APPLICATIONS

This application is a continuation of Ser. No. 13/899,562 filed May 21, 2013 which claims benefit of priority to U.S. Provisional Patent Application No. 61/650,253, filed May 22, 2012, and this applications claims benefit of priority to U.S. Provisional Patent Application No. 61/667,584, filed Jul. 3, 2012, all of which are hereby incorporated by reference as if fully set forth.

### FIELD OF THE DISCLOSURE

This disclosure relates to the detection of alterations in KRAS expression in a subject with colorectal cancer treated with anti-EGFR therapy. Methods for detection of the alterations, identification of resistance of the colorectal cancer to the therapy, and treatment to prevent, reverse, or delay the resistance are also disclosed.

### BACKGROUND OF THE DISCLOSURE

A main limitation of therapies that selectively target kinase signaling pathways is the emergence of secondary drug resistance. Cetuximab, a monoclonal antibody that binds the extracellular domain of EGFR, is effective in a subset of KRAS wild type metastatic colorectal cancers (Ciardiello, F. & Tortora, G. EGFR antagonists in cancer treatment. N Engl J Med 358, 1160-1174 (2008). After an initial response, secondary resistance invariably ensues, thereby limiting the clinical benefit of this drug (Karapetis, C. S. et al. K-ras mutations and benefit from cetuximab in advanced colorectal cancer. N Engl J Med 359, 1757-1765 (2008). The molecular bases of secondary resistance to cetuximab in colorectal cancer are poorly understood (Wheeler, D. L. et al. Mechanisms of acquired resistance to cetuximab: role of HER (ErbB) family members. Oncogene 27, 3944-3956 (2008); Benavente. S. et al. Establishment and characterization of a model of acquired resistance to epidermal growth factor receptor targeting agents in human cancer cells. Clin. Cancer Res. 15, 1585-1592, doi:10.1158/1078-0432.CCR-08-2068 (2009); Li, C., Iida, M., Dunn, E. F., Ghia, A. J. & Wheeler, D. L. Nuclear EGFR contributes to acquired resistance to cetuximab. Oncogene (2009); Hatakeyama, H. et al. Regulation of heparin-binding EGF-like growth factor by miR-212 and acquired cetuximab resistance in head and neck squamous cell carcinoma. PLoS One 5, e12702, doi:10.1371/journal.pone.0012702 (2010): Yonesaka, K. et al. Activation of ERBB2 signaling causes resistance to the EGFR-directed therapeutic antibody cetuximab. Science translational medicine 3, 99ra86, doi: 10.1126/scitranslmed.3002442 (2011); Montagut, C. et al. Identification of a mutation in the extracellular domain of the Epidermal Growth Factor Receptor conferring cetuximab resistance in colorectal cancer. Nature medicine, doi:10.1038/nm.2609 (2012).

The citation of documents herein is not to be construed as reflecting an admission that any is relevant prior art. Moreover, their citation is not an indication of a search for relevant disclosures. All statements regarding the date(s) or contents of the documents is based on available information and is not an admission as to their accuracy or correctness.

### BRIEF SUMMARY OF THE DISCLOSURE

The disclosure relates to methods of predicting if a subject being treated for colorectal cancer (CRC) with anti-EGFR therapy will develop drug resistance. One method comprises obtaining a biological sample from the subject, assaying the sample for an alteration in KRAS expression, wherein if there is an alteration in KRAS expression, the subject is more likely to develop drug resistance to anti-EGFR therapy.

The alteration in KRAS expression may be the expression of a KRAS mutant (somatic mutation), increased KRAS gene or protein expression (focal amplification) or increased KRAS activation, when compared to a control (non-cancerous) sample. Embodiments of the KRAS mutation include G13D, G12R, Q61H or A146T mutation as non-limiting examples.

The anti-EGFR therapy may be treatment with cetuximab or panitumumab or other antibody-based therapies as non-limiting examples. The biological sample may be blood, plasma, serum, urine, tissue, cells or a biopsy as non-limiting examples.

The present disclosure also provides methods of preventing, reducing or delaying the onset of drug resistance to anti-EGFR therapy as described herein. One method comprises administering, to a subject having an alteration in KRAS expression, an MEK inhibitor in combination with the anti-EGFR therapy. In some embodiments, the subject is afflicted with, or been diagnosed with, colorectal cancer.

The alteration in KRAS expression may be the expression of a KRAS mutant, increased KRAS gene or protein expression, or increased KRAS activation as disclosed herein. Non-limiting examples of an MEK inhibitor include XL 518, CI-I040, PD035901, GSK1120212 or selumetinib. The anti-EGFR therapy may be treatment with cetuximab or panitumumab or other antibody-based therapies as non-limiting examples. The biological sample may be blood, plasma, serum, urine, tissue, cells or a biopsy as non-limiting examples.

The patent and scientific literature referred to herein establishes the knowledge that is available to those with skill in the art. Genbank and NCBI submissions indicated by accession number cited herein are hereby incorporated by reference.

While this disclosure has been particularly shown and described with references to preferred embodiments thereof, it will be understood by those skilled in the art that various changes in form and details may be made therein without departing from the scope of the disclosure encompassed by the appended claims.

### BRIEF DESCRIPTION OF THE FIGURES

FIGS. 1A-F show that KRAS amplification mediates acquired resistance to cetuximab in DiFi cells. (FIG. 1A) Parental and cetuximab resistant DiFi cells were treated for one week with increasing concentrations of cetuximab. Cell viability was assayed by the ATP assay. Data points represent means±SD of three independent experiments. (FIG. 1B) Whole exome gene copy number analysis of parental and cetuximab resistant DiFi cells. Individual chromosomes are indicated on the x axis. The lines indicate the sequencing depth (y axis) over exome windows of 100,000 bp. (FIG. 1C) FISH analysis confirming KRAS amplification in DiFi-R but not parental DiFi cells. KRAS locus BAC DNA (probe RPII-707G18; green) and chromosome 12 paint (red) were hybridized to the metaphase spreads of DiFi cells. (FIG. 1D) DiFi cells were treated with cetuximab 35 nM for 24 hours, after which whole-cell extracts were subjected to Western blot analysis and compared to untreated cells. DiFi R1 and R2 were plated in the absence of cetuximab for 7 days or maintained in their normal growth medium (with cetuximab 35 nM) before protein analysis. Active KRAS (GTP-KRAS) was assessed by GST-Raf1 pull-down. Whole-cell extracts were blotted with phosphor-EGFR (Tyr 1068), total EGFR, total KRAS, phosphor-AKT (Thr 308), phosphor-AKT (Ser473), total AKT, total MEK1/2 and phospho-MEK1/2, total ERK1/2 and phospho-ERK1/2 antibodies. Vinculin was included as a loading control. (FIG. 1E) Western blot analysis of KRAS protein in DiFi cells infected with a KRAS lentivirus. Actin is shown as a loading control. (FIG. 1F) Ectopic expression of wild-type KRAS in parental DiFi cells confers resistance to cetuximab. Data points represent means±SD of three independent experiments.

FIGS. 2A-E show that KRAS mutations mediate acquired resistance to cetuximab in Lim1215 cells. (FIG. 2A) Parental and cetuximab resistant Lim1215 cells were treated for one week with increasing concentrations of cetuximab. Cell viability was assayed by the ATP assay. Data points represent means±SD of three independent experiments. (FIG. 2B) Sanger sequencing of KRAS exon 2 in parental and two representative cetuximab-resistant Lim1215 cells obtained in independent selection procedures. (FIG. 2C) Western blot analysis of the EGFR signaling pathway in parental and cetuximab resistant Lim1215 cells. (FIG. 2D) Schematic representation of the vectors used to knock-in the G12R and G13D mutations into the genome of Lim1215 parental cell lines by AAV mediated homologous recombination. Targeting was assessed by Sanger sequencing. (FIG. 2E) Parental and isogenic Lim1215 cells carrying the indicated mutations were treated for one week with increasing doses of cetuximab. Data points represent means±SD of three independent experiments.

FIGS. 3A-C show mutational analysis of the KRAS gene in patients. (FIG. 3A) Mutational analysis of KRAS in chemorefractory patients. (FIG. 3B) Mutational analysis of the KRAS gene in patients who progressed on anti-EGFR antibodies. The results are based on assays performed by Deep sequencing technologies a: 454 pyrosequencing; b: BEAMing. (FIG. 3C) Dot plot of percentage of mutated KRAS alleles in chemorefractory and anti-EGFR resistant patients: p-value was calculated by two-tailed unpaired Mann-Whitney test.

FIGS. 4A-D show detection of circulating KRAS mutant DNA in a patient with acquired resistance to cetuximab therapy. (FIG. 4A) Size of liver metastasis (blue bars) and CEA levels in blood (blue line) at the indicated time points showing an initial response to cetuximab followed by progression (Patient 8). (FIG. 4B) Quantitative analysis of Q61H mutant DNA in plasma as assessed by BEAMing (green line). (FIG. 4C) Two dimensional dot plot showing quantitative analysis of the KRAS Q61H mutation in plasma using BEAMing at individual time points. (FIG. 4D) Mutational analysis of KRAS on tumor samples collected pre-cetuximab treatment and at the time of disease progression.

FIGS. 5A-C show sensitivity to cetuximab of the DiFi and LIM1215 parental cell lines. (FIG. 5A) The indicated cell lines were treated for one week with increasing doses of cetuximab. Cell viability was assayed by the ATP assay. Data points represent means±SD of three independent experiments. (FIG. 5B) Western blot analysis of EGFR expression levels in DiFi and Lim1215 cells. (FIG. 5C) FISH analysis of the EGFR gene in DiFi and Lim1215 cell lines. Red EGFR gene probe; Green Chr 7 centromeric probe.

FIG. 6 shows a schematic representation of the strategy used to derive cetuximab resistant cell lines. The concentrations of drug and the protocols (constant and incremental) are illustrated.

FIGS. 7A-C show acquired resistance to cetuximab is associated with focal amplification of the KRAS locus in DiFi cells. (FIGS. 7A and 7B) High resolution analysis of EGFR and KRAS amplicons in parental and cetuximab resistant DiFi cells. Dots represent exon-averages while segments are gene-averages of the sequencing depth (blue: parental DiFi; red: resistant DiFi). (FIG. 7C) The number of copies corresponding to the EGFR and KRAS loci was determined by real-time quantitative PCR using gDNA extracted from DiFi parental, R1 and R2 cells. Primers designed to span centromeric regions of chromosomes 7 or 12 were employed to normalize data for aneuploidy. Genomic DNA from a diploid cell line (HCEC) was used as a reference control. Histograms represent means±SD of three independent experiments.

FIG. 8 shows KRAS amplified cells are present in DiFi cells before cetuximab treatment. Immunostaining of KRAS protein in DiFi parental and resistant cells shows the presence of KRAS over expressing cells in the parental population.

FIGS. 9A-E show KRAS mutations can arise ‘de novo’ during cetuximab treatment. (FIG. 9A) Schematic representation of the protocol used to obtain a KRAS wild type Lim1215 clone and to derive its cetuximab resistant variants. (FIG. 9B) Mass Spectrometry analysis of Lim1215 E4.1 cetuximab resistant cells showing the KRAS nucleotide change at codon 146 (G436A). (FIG. 9C) Proliferation of the Lim1215 KRAS WT subclone E4.1 is impaired by cetuximab treatment. (FIG. 9D) KRAS A146T resistant cells derived from the E4.1 subclone are fully insensitive to cetuximab. In FIGS. 9C and 9D, error bars represent SD of six technical replicates; the assay was performed three times with comparable results. (FIG. 9E) KRAS A146T cetuximab resistant cells display active GTP-RAS, as assessed by GST-Raf1 pull-down. Whole-cell extracts were blotted total RAS antibody, while GAPDH was included as a loading control.

FIGS. 10A-F show combinatorial inhibition of EGFR and MEK is effective in cells with acquired resistance to cetuximab. (FIGS. 10A-10D) Cetuximab-resistant DiFi or Lim1215 cells were treated with a constant dose of cetuximab (70 nM) and/or with increasing doses of the PI3K inhibitor GSK1059615 (FIGS. 10A and 10B) or MEK inhibitor AZD6244 (FIGS. 10C and 10D) for one week. Cell viability was assayed by the ATP assay. Data points represent means±SD of three independent experiments. (FIGS. 10E and 10F) Western blot analysis of phosphorylated ERK expression and activation in the indicated cell lines treated with cetuximab (350 nM for DiFi R2 and 1400 nM for Lim1215 R2) and AZD6244 1 μM.

FIGS. 11A-D show acquired resistance to cetuximab is associated with focal amplification of the KRAS locus in colorectal tumors. (FIGS. 11A and 11B) Immunohistochemical analysis of KRAS protein expression in tumor tissues before (PR) and after (PD) development of resistance to cetuximab. (FIGS. 11C and 11D) FISH analysis of the KRAS gene in the same patient. Red Chr12 centromeric probe; Green KRAS gene probe.

### DETAILED DESCRIPTION OF MODES OF PRACTICING THE DISCLOSURE

### General

The disclosure provides methods and compositions for detecting and predicting resistance to anti-EGFR treatment in colorectal cancers. Also provided are methods and compositions for preventing, reversing or delaying the resistance. The present disclosure also provides kits for use in the methods described herein.

Methods

As described herein, the disclosure includes methods for detecting or identifying the presence of resistance to anti-EGFR treatment in a colorectal cancer. The methods may include detecting or identifying the presence of an alteration or change in KRAS expression in a biological sample obtained from a subject. In some cases the subject may be afflicted with, or diagnosed with, a colorectal cancer. Additionally, the subject may be human, such as a patient under clinical care.

In some embodiments, the biological sample may be a body fluid, such as blood, plasma, serum, or urine from the subject. In some cases, the fluid is cell-free. In other embodiments, the sample may contain tissue or cells from the subject. In some cases, the sample is a cell-containing biopsy from the subject, such as a formalin fixed paraffin embedded (FFPE) sample. A biological sample for use in the disclosure contains nucleic acid molecules encoding KRAS protein or a disclosed mutant form thereof. In some embodiments, the nucleic acid molecules are DNA, cDNA, or RNA oligonucleotides, or fragments of cellular nucleic acids, that encode all or part of a KRAS protein. In some cases, the oligonucleotide encodes a portion of the KRAS protein containing a mutation disclosed herein in relation to resistance to anti-EGFR treatment. The KRAS mutations include G13D, G12R, Q61H, or A146T in the human KRAS amino acid sequence of SEQ ID NO: 2 or 4.

Methods for the analysis of a body fluid are known to the skilled person and may be applied to the detection of an alteration or change in KRAS expression as disclosed herein. Non-limiting examples include methods of assaying nucleic acid molecules, including genomic DNA sequences and expressed RNA sequences as non-limiting examples, and methods of assaying proteins and polypeptides, including antibody-based detection, western blotting, and mass spectroscopy as non-limiting examples. In cases of a sequence in an RNA molecule, a skilled person may first prepare a corresponding cDNA molecule by well-known methods in the field. Alternatively, the skilled person may use methods that utilize the RNA molecule directly. In some embodiments, the methods may be applied to detect the amount, or level, of KRAS expression. This includes, but is not limited to, the amount of expression of a KRAS mutation. In other embodiments, the methods may be applied to detect a KRAS mutation as disclosed herein.

Using a plasma or urine sample as a non-limiting exemplar, the presence of a KRAS mutation in the nucleic acid molecules present in the sample may be detected by known methods such as PCR amplification and/or sequencing as non-limiting examples. In the case of a plasma sample, the nucleic acid molecules include those released from at least one cancer cell with a KRAS mutation. In the case of a urine sample, the nuclei acid molecules include transrenal molecules that have passed across the kidney bather from a subject's blood into the subject's urine. In some embodiments to detect a KRAS DNA sequence in a nucleic acid molecule in the sample, DNA molecules may be isolated by use of known methods and then amplified, optionally by PCR (polymerase chain reaction) with at least one primer, to produce large numbers of an oligonucleotide “amplicon” containing a sequence with a KRAS mutation. The at least one primer may be complementary to all or part of an oligonucleotide containing a KRAS sequence.

In other embodiments, the oligonucleotide contains a contiguous sequence containing both a KRAS sequence and one or more exogenous sequences not covalently linked to KRAS sequences in nature. Non-limiting examples of an “amplicon” include the presence of an adapter sequence at one or both ends of the oligonucleotide containing a KRAS sequence. The amplified oligonucleotide may optionally be detectably labeled during, or after, the amplification process. In further embodiments, the amplified oligonucleotide may be sequenced to detect the presence of a mutation in the KRAS sequence. In some cases, a high-throughput or massively parallel DNA sequencing method may be used; non-limiting examples include GS FLX by 454 Life Technologies/Roche, Genome Analyzer by Solexa/Illumina, and SOLiD by Applied Biosystems. Alternative sequencing embodiments include CGA Platform by Complete Genomics, and PacBio RS by Pacific Biosciences, semiconductor-based sequencing by Life Technologies and Ion Torrent, and nanopore-based sequencing. The KRAS sequence may be of a convenient length, such as about 25, about 50, about 75, or about 100 or more nucleotides or more, that encodes a mutation selected from G13D, G12R, Q61H, and A146T in the human KRAS amino acid sequence of SEQ ID NO: 2 or 4.

In the case of detecting an RNA sequence in the sample, RNA encoding KRAS protein may be isolated by use of known methodology and then converted into a cDNA molecule, such as an oligonucleotide. Using the cDNA form, the detection process may proceed as disclosed above for DNA molecules. This includes a cDNA that contains a KRAS mutation as disclosed herein. And in a case of a tissue, cell, or biopsy sample, the sample may be processed to isolate DNA or RNA by methods known to the skilled person. The isolated DNA and/or RNA may then be used to detect a KRAS mutation as disclosed herein.

Therefore, the disclosure includes a method of detecting a KRAS mutation in a sample of blood, plasma, serum, or urine from a subject, the method comprising preparation of cell-free DNA or RNA from the sample and detection of the presence of a KRAS mutation selected from G13D, G12R, Q61H, and A146T in the human KRAS amino acid sequence of SEQ ID NO: 2 or 4. In some embodiments, the detection comprises the amplification and/or sequencing of a sequence of about 25, about 50, about 75, or about 100 or more nucleotides that contains a sequence containing a KRAS mutation. As disclosed herein, the presence of one of these mutations indicates that a subject with colorectal cancer will develop resistance to treatment with anti-EGFR therapy. As disclosed herein, the presence of the mutation may be due to its pre-existence in one or more tumor cells of the colorectal cancer or due to mutation during treatment.

The disclosure further includes a method of predicting if a subject, such as a human patient, treated for colorectal cancer with anti-EGFR therapy will develop drug resistance. The anti-EGFR therapy may be treatment with cetuximab or panitumumab as non-limiting examples. The method may comprise obtaining a biological sample of this disclosure from a subject and assaying the sample for an alteration in KRAS expression. In some embodiments, the detection of an alteration in KRAS expression indicates or predicts that the subject is more likely to develop drug resistance to the anti-EGFR therapy. In some cases, the alteration is the expression of a mutant KRAS polypeptide due to a mutation of the coding sequence. The disclosure includes coding sequence mutations that result in a mutation selected from G13D, G12R, Q61H, and A146T in the human KRAS amino acid sequence of SEQ ID NO: 2 or 4.

In other embodiments, a skilled person may assay for, or determine, an alteration in KRAS expression by comparing the expression of KRAS in a biological sample from a subject to the expression of KRAS in a control, non-cancerous biological sample from the same subject or another subject. In some cases, the alteration in the KRAS expression is an increase in nucleic acid or protein expression of KRAS when compared to the control, non-cancerous biological sample. The increase in nucleic acid or protein expression indicates, or predicts, that the subject treated with anti-EGFR therapy will develop drug resistance. In other cases, the alteration in the KRAS expression is a decrease in nucleic acid or protein expression of KRAS when compared to the control, non-cancerous biological sample.

In further embodiments, a skilled person may assay for, or determine, an alteration in KRAS expression by comparing the expression of KRAS, in the form of KRAS functional activity, in a biological sample from a subject to that in a control, non-cancerous biological sample from the same subject or another subject. In some cases, the alteration in the KRAS expression is an increase in functional activity when compared to the control, non-cancerous biological sample. The increase in functional activity indicates, or predicts, that the subject treated with anti-EGFR therapy will develop drug resistance. In other cases, the alteration in the KRAS expression is a decrease in functional activity when compared to the control, non-cancerous biological sample.

The disclosure additionally includes a method of preventing, reducing or delaying the onset of resistance to anti-EGFR therapy, such as in a subject or patient with a disclosed alteration in KRAS expression. The subject or patient is optionally previously diagnosed with colorectal cancer. The anti-EGFR therapy may be treatment with cetuximab or panitumumab as non-limiting examples disclosed herein. The method may comprise administering, to a subject with a disclosed alteration in KRAS expression, a MEK inhibitor in combination with anti-EGFR therapy. The MEK inhibitor may be XL 518, CI-I040, PD035901, GSK1120212 or selumetinib as non-limiting examples. In some embodiments, the alteration in KRAS expression is the expression of a KRAS mutant as described herein. In some cases, expression of a KRAS mutant is in a biological sample of blood, plasma, serum, urine, tissue, cells or a biopsy as disclosed herein. Of course the detection of the KRAS mutant may be by any appropriate method described herein.

In other embodiments, the alteration in KRAS expression is determined by comparing the expression of KRAS in a biological sample from the subject being treated with anti-EGFR therapy to the expression of KRAS in a control, non-cancerous biological sample as described above. In some cases, the alteration in KRAS expression is an increase, or a decrease, in nucleic acid or protein expression when compared to the control, non-cancerous biological sample as described herein. In other cases, the alteration in KRAS expression is an increase, or decrease, in KRAS functional activity when compared to said control, non-cancerous biological sample as described herein.

Compositions and Kits

The disclosure includes compositions and kits for use in the methods disclosed herein. Non-limiting examples of a composition or reagent include at least one PCR and/or sequencing primer for use in the detection of a KRAS mutation as disclosed herein. In some embodiments, an oligonucleotide primer may be of about 18, about 20, about 22, about 24, or about 26 or more nucleotides that is complementary to all of a KRAS nucleic acid sequence within about 25, about 50, about 75, or about 100 or more nucleotides of a mutation that results in a mutation selected from G13D, G12R, Q61H, and A146T in the human KRAS amino acid sequence of SEQ ID NO: 2 or 4. In some cases, the primer may be complementary to the nucleotide(s) that are mutated in the KRAS nucleic acid sequence. In other embodiments, the primer may contain a mismatch to the nucleotide(s) that are mutated in the KRAS nucleic acid sequence. The skilled person is aware of methods for using primers with no mismatch or with a mismatch to detect the presence of a sequence of interest, such as a mutated KRAS nucleic acid sequence.

A composition or reagent may be part of a kit of components, optionally with instructions and/or labels, for performance of a method disclosed herein. Optional other reagents buffers, nucleotides, and enzymes for use in nucleic amplification and/or sequencing as described herein. Other reagents include nuclease inhibitors for use in the collection of a biological sample, such as a blood or urine sample; components for the preparation of cell-free samples and nucleic acids; and components for the lysis of cells to release nucleic acids.

### Additional Embodiments

The present invention provides compositions and methods for detecting molecular alterations (in most instances point mutations) of KRAS that are causally associated with the onset of acquired resistance to anti-EGFR treatment in colorectal cancers. Expression of mutant KRAS under the control of its endogenous gene promoter is sufficient to confer cetuximab resistance but resistant cells remained sensitive to combinatorial inhibition of EGFR and MEK. Analysis of metastases from patients who developed resistance to cetuximab or panitumumab showed the emergence of KRAS amplification in one sample and acquisition of secondary KRAS mutations in 60% (6/10) of the cases. KRAS mutant alleles were detectable in the blood of cetuximab treated patients as early as 10 months prior to radiographic documentation of disease progression. The results provided herein identify KRAS mutations as frequent drivers of acquired resistance to cetuximab in colorectal cancers, indicate that the emergence of KRAS mutant clones can be detected non-invasively months prior to radiographic progression and suggest early initiation of a MEK inhibitor as a rational strategy for delaying or reversing drug resistance.

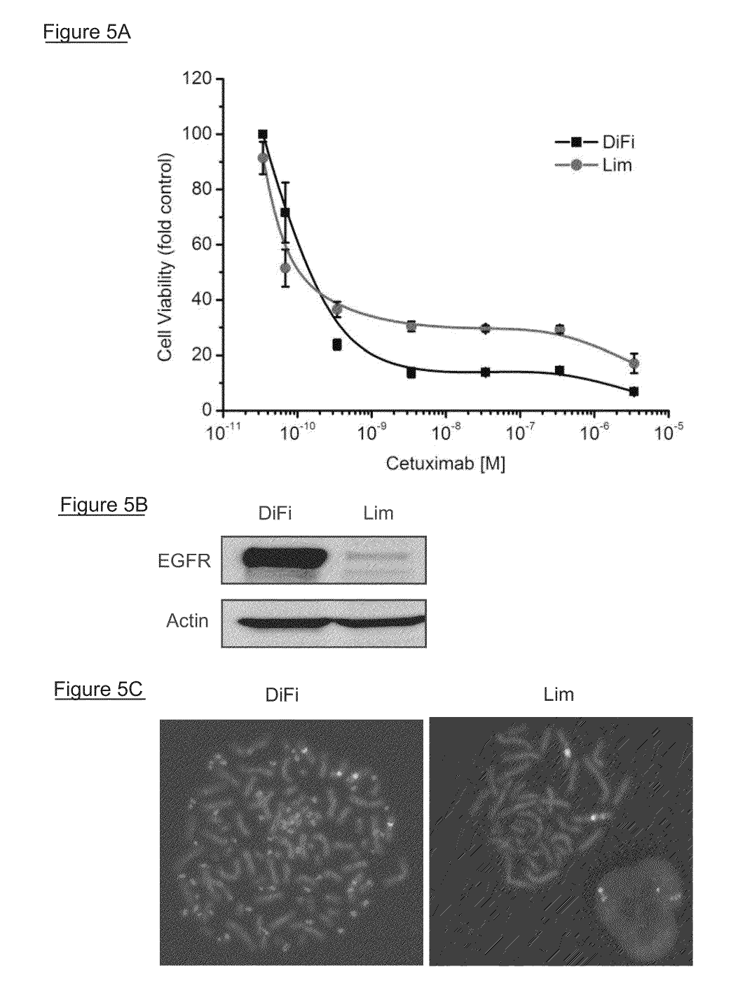

Defining the molecular bases of secondary resistance to anti-EGFR therapies is critical to monitor, prevent and/or overcome drug refractoriness. To identify potential mechanisms of cetuximab resistance, cetuximab-resistant variants of two colorectal cancer (CRC) cellular models were generated (DiFi, Lim1215) that are highly sensitive to EGFR inhibition (FIG. 5a). DiFi cells overexpress EGFR as a result of high level amplification of the EGFR gene locus (Moroni, M. et al. Gene copy number for epidermal growth factor receptor (EGFR) and clinical response to antiEGFR treatment in colorectal cancer: a cohort study. The Lancet Oncology 6, 279-286, doi:10.1016/S1470-2045(05)70102-9 (2005)). In contrast, Lim1215 cells express ‘normal’ levels of EGFR but are similarly sensitive to cetuximab (FIGS. 5b, 5c). Both cell lines are wild type for KRAS, BRAF and PIK3CA, paralleling the molecular features of the CRC patients most likely to respond to cetuximab (De Roock W. et al. Effects of KRAS, BRAF, NRAS, and PIK3CA mutations on the efficacy of cetuximab plus chemotherapy in chemotherapy-refractory metastatic colorectal cancer: a retrospective consortium analysis. Lancet Oncol. 11, 753-762 (2010).

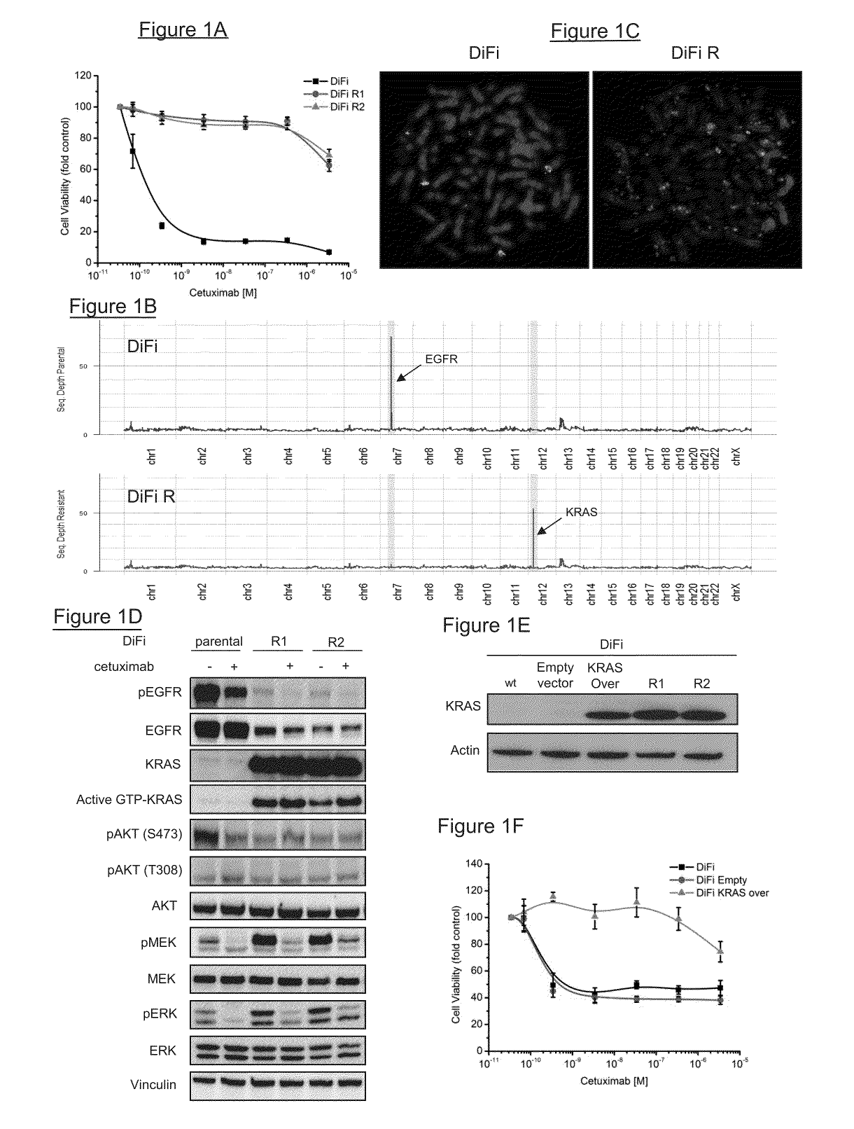

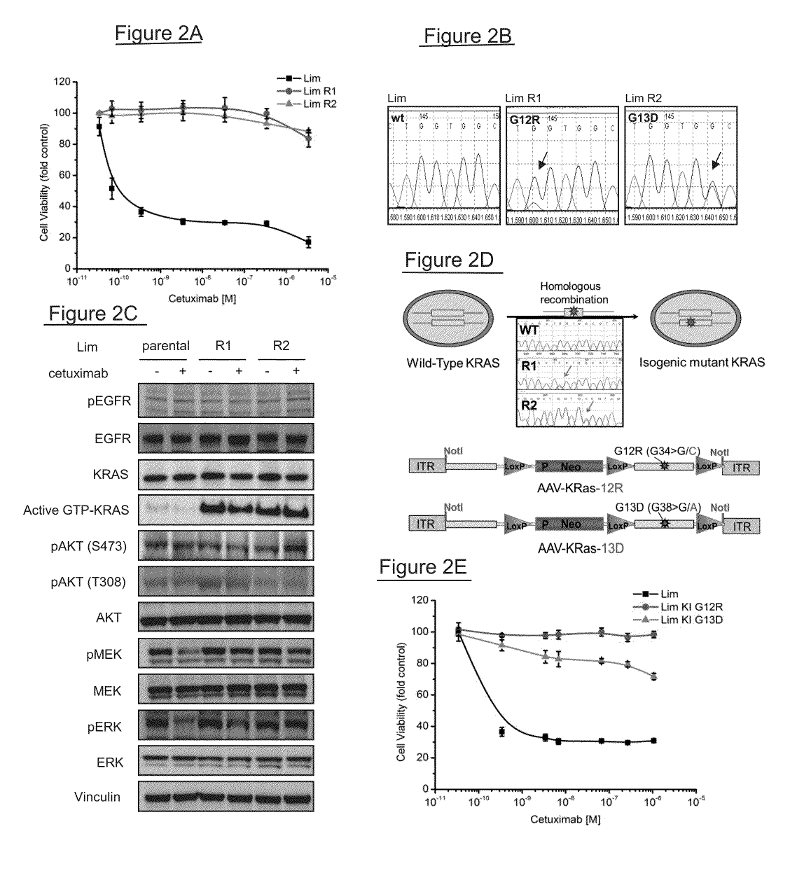

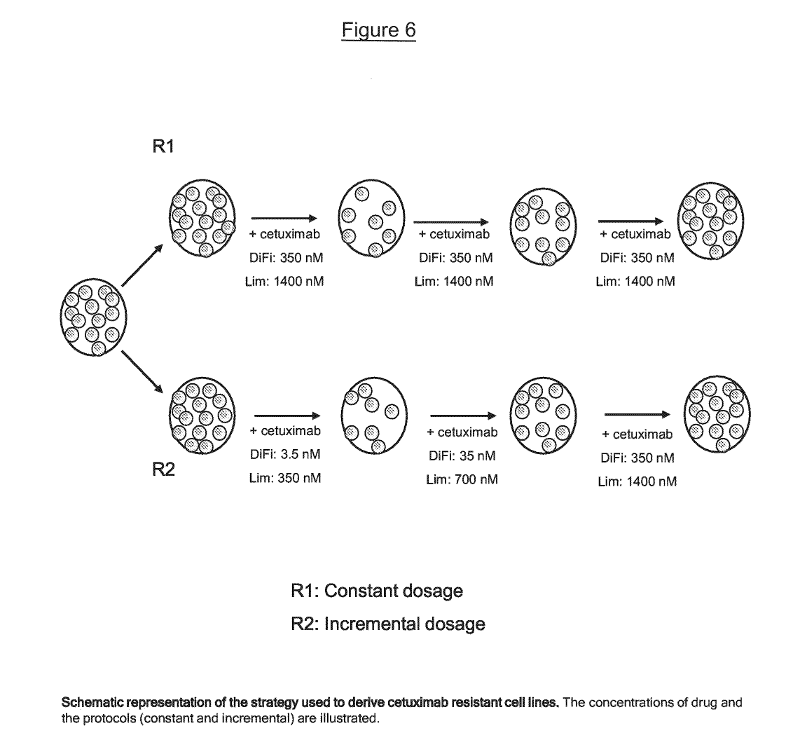

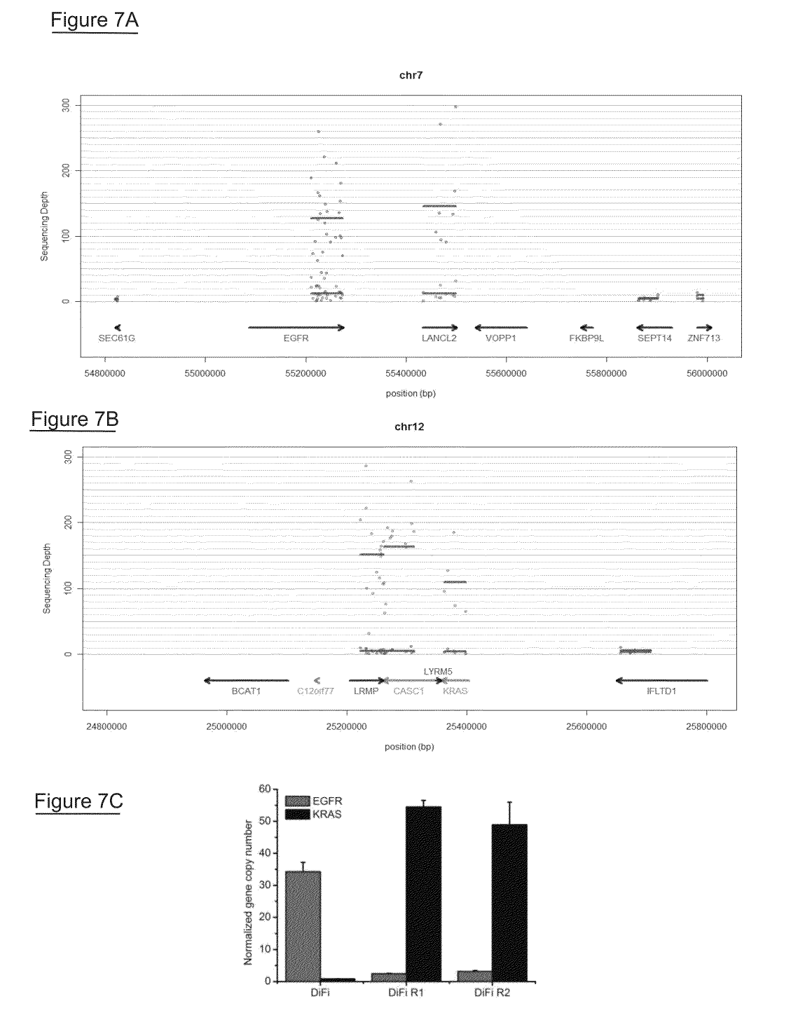

Continuous drug treatment using two different protocols (see Example methods and FIG. 6) led to the emergence of cetuximab resistant variants (DiFi-R and Lim1215-R, FIGS. 1a, 2a). To identify the molecular basis of cetuximab resistance in these cells, gene copy number analysis and mutational profiling of the resistant and parental lines was performed. Cetuximab-resistant DiFi-R cells differed from their sensitive parental counterpart by two focal molecular alterations: EGFR gene copy number was reduced whereas the KRAS gene was amplified (FIGS. 1b, 1c, 7). These genomic changes were accompanied by reduced EGFR and increased KRAS protein expression in the cetuximab resistant cells (FIG. 1d). Sequence analysis confirmed that the EGFR, KRAS, NRAS, HRAS, BRAF and PIK3CA genes were wild type in the cetuximab-resistant clones.

Sequence analysis of the Lim1215 cetuximab resistant variants identified acquisition of either G3D or G12R KRAS mutations (FIG. 2b). In both DiFi-R and Lim1215-R cells, KRAS amplification or mutations, respectively, were accompanied by increased KRAS activation relative to their parental counterparts. In the presence of KRAS amplification, cetuximab could partially abrogate phosphorylation of MEK and ERK but, like in KRAS mutant cells, was unable to induce growth arrest (FIGS. 1a, 1d, 2a, 2e).

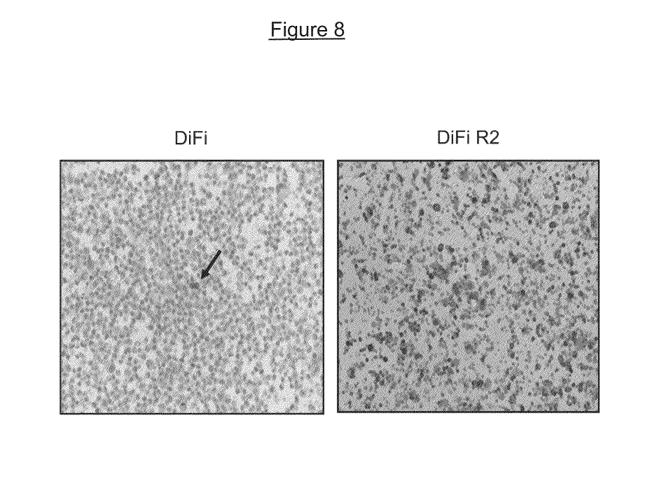

To determine whether resistance was due to selection of pre-existing drug resistant cells, the parental cell lines were analyzed in depth for the presence of a minority population of KRAS amplified or mutant cells. In the parental DiFi cells, a sub-population with high level KRAS amplification was identified at a prevalence of approximately 1:40,000 (FIG. 8). Similarly, deep sequencing and BEAMing (Bead Emulsion Amplification and Magnetics) (Diehl, F. et al. Circulating mutant DNA to assess tumor dynamics. Nat Med 14, 985-990 (2008) indicated that approximately 0.2% of the parental Lim1215 cells harbored the KRAS GI3D mutation (Table 1).

TABLE 1

| KRAS | | | |
|---|---|---|---|
| Mutations | Lim | Lim R1 | Lim R2 |
| G12R | 0% | 20% | 0% |
| G13D | 0.22% | 0% | 47% |

Specifically, Table 1 shows that KRAS mutant cells are present in Lim1215 cells before cetuximab treatment. In the table, percentage of KRAS mutant alleles in parental and cetuximab resistant Lim1215 cells as measured by BEAMing. Notably, the G12R mutation was not detectable in the earliest available passage of parental cells, even when the analysis was performed at high coverage (>50,000 fold). These results indicate that the emergence of a cetuximab resistant population could derive from selection of a pre-existing KRAS amplified or mutant clone, or as the result of ‘de novo’ acquisition of a KRAS mutation under the pressure of cetuximab treatment. To formally assess this latter possibility, dilution cloning of the earliest available passage of Lim1215 cells was performed in order to generate a homogenous, KRAS wild-type Lim1215 subline.

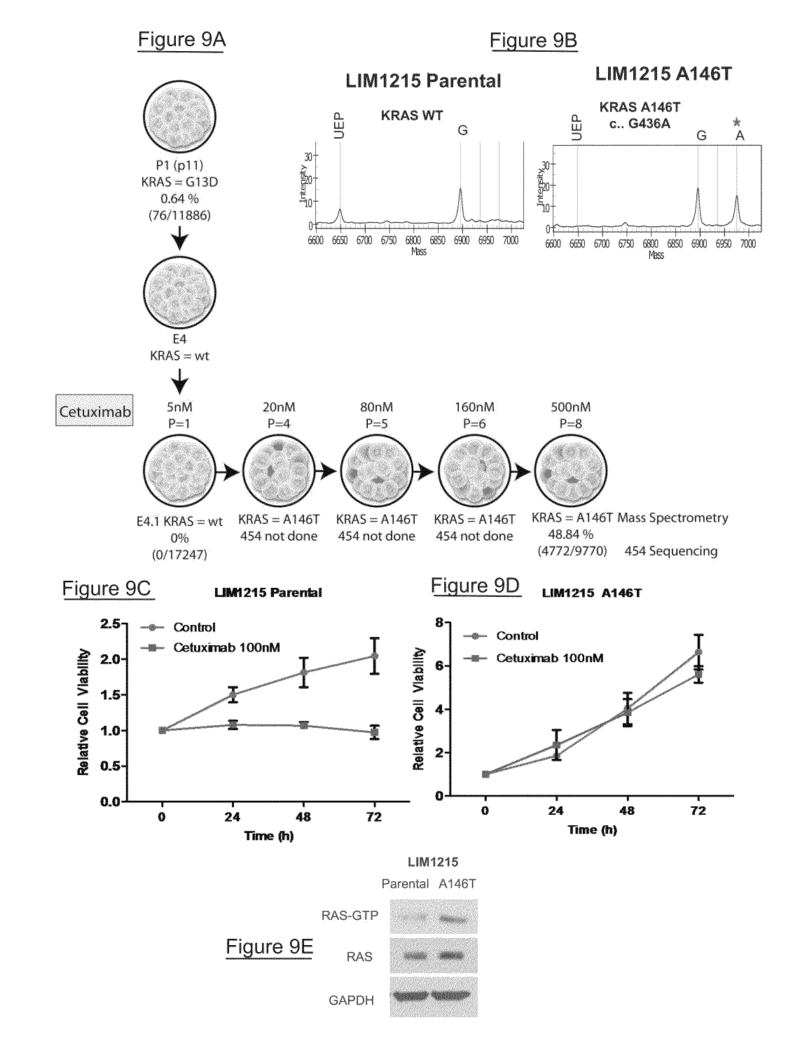

As schematized in FIG. 9, two successive dilution cloning experiments were performed and the derivative cells (hereafter referred to as E4.1) were confirmed as KRAS wild-type by both mass spectrometry (MS) based genotyping and by 454 analysis. The E4.1 cells were then cultured in increasing concentrations of cetuximab, analogous to the experiment performed with the original Lim1215 parental line. Cells were collected during intermediate passages and subjected to MS based genotyping and/or 454 analysis (FIG. 9a). MS genotyping identified a KRAS A146T mutation following four passages in increasing concentrations of cetuximab (20 nM and higher. FIG. 9b). These cells were indeed resistant to the drug (FIGS. 9c, 9d) and displayed biochemical activation of KRAS (FIG. 9e). In parallel, genetic analysis of the E4.1 cells grown in medium without cetuximab found them to be KRAS wild-type. In sum, these data indicate that resistance to cetuximab in Lim1215 cells may emerge not only from the selection of pre-existing KRAS mutant clones but also as a result of ongoing mutagenesis.

To validate that amplification or mutations of KRAS were causally responsible for cetuximab resistance in the in vitro models, two sets of forward genetic experiments were performed. First, ectopic overexpression of wild type KRAS in DiFi conferred resistance to cetuximab (FIGS. 1e, 1f). Second, AAV-mediated targeted homologous recombination was employed to introduce (knock-in) the G13D and G12R alleles into the endogenous KRAS locus of Lim1215 cells (Di Nicolantonio, F. et al. Replacement of normal with mutant alleles in the genome of normal human cells unveils mutation-specific drug responses. Proc. Natl. Acad Sci. USA. 105, 20864-20869, doi:10.1073/pnas.0808757105 (2008). Knock-in of the G13D or G12R mutant alleles rendered Lim1215 cells resistant to cetuximab (FIGS. 2d, 2e).

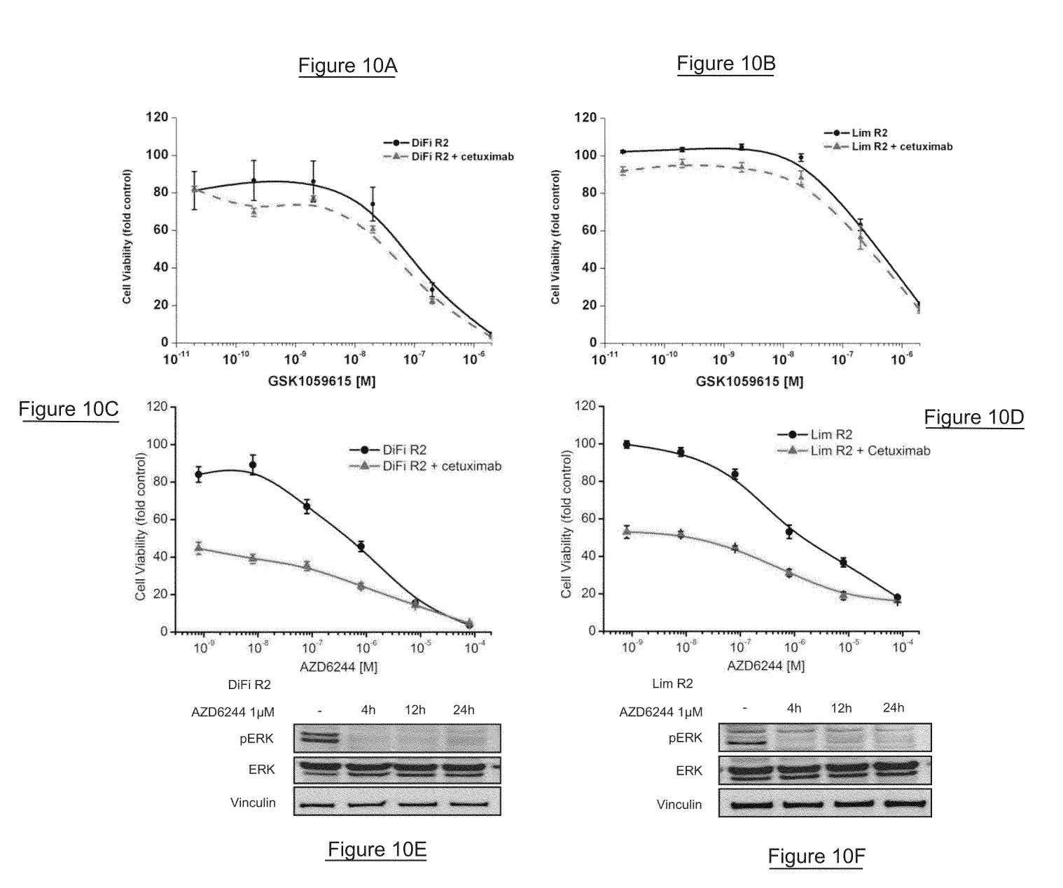

Chemotherapy-refractory CRC patients who initially respond and then become resistant to cetuximab have no further therapeutic options. It was reasoned that cetuximab resistance resulting from constitutive KRAS activation could be prevented or reversed by pharmacologic inhibition of KRAS signaling. Thus, the resistant clones were co-treated with cetuximab and selective inhibitors of the MEK and PI3 kinases, two key downstream effectors of oncogenic KRAS. While inhibitors of PI3 kinase were ineffective in the cetuximab resistant cells, both the Lim1215-R and DiFi-R cells were sensitive to combinatorial targeting of MEK and EGFR (FIG. 10).

To determine whether KRAS mutation and/or amplification are clinically relevant mechanisms of acquired cetuximab-resistance, tumor biopsies from 10 CRC patients who had become refractory to either cetuximab or the anti-EGFR antibody panitumumab were examined (see Table 2, which describes the Patients' clinical characteristics).

TABLE 2

| | | | | | | | | | Site of | Site of | | | |
|---|---|---|---|---|---|---|---|---|---|---|---|---|---|
| | | | | | | | | | mutational | mutational | | BRAF | |
| | | Site of | Anti-EGFR treatment | Irinotecan | Best | Duration of | Time of biopsy | | analysis anti- | analysis anti- | FFPE/ | mutational | PIK3CA |
| Patient ID | Gender | primary tumor | monoclonal antibody/CT | refractory | response | treatment | after progression | | EGFR sensitive | EGFR resistant | Frozen | status | mutational status |
| Patient #1 | M | rectum | panitumumab + | yes | SD | 20 months | 6 | months | rectum | liver | FFPE | wt (exon 15) | wt (exons 9-20) |
| | | | irinotecan | | | | | | | | | | |
| Patient #2 | M | ascending | panitumumab + | yes | PR | 6 months | 12 | months | liver | R chest wall | FFPE | wt (exon 15) | wt (exons 9-20) |
| | | colon | irinotecan | | | | | | | subcut nodule | | | |
| Patient #4 | F | sigmoid colon | cetuximab + irinotecan | no | SD | 5 months | 7 | months | colon | lung | FFPE | wt (exon 15) | wt (exons 9-20) |
| Patient #5 | F | sigmoid colon | cetuximab + irinotecan | yes | PR | 7 months | 1 | month | colon | liver | FFPE | wt (exon 15) | wt (exons 9-20) |
| Patient #6 | M | cecum | cetuximab + FOLFIRI | no | PR | 21 months | 14 | months | liver | liver | FFPE | wt (exon 15) | wt (exons 9-20) |
| Patient #7 | M | sigmoid colon | cetuximab + irinotecan; | no | SD | 25 months | 1 | month | liver | cerebellum | FFPE | wt (exon 15) | not done |
| | | | panitumumab + FOLFIRI | | | | | | | | FFPE | wt (exon 15) | wt (exons 9-20) |
| Patient #8 | M | sigmoid colon | cetuximab + irinotecan | yes | PR | 18 months | 1 | month | liver | liver | FFPE | wt (exon 15) | wt (exons 9-20) |
| Patient #9 | M | rectum | cetuximab + irinotecan | yes | PR | 20 months | 1 | month | liver | liver | FFPE | wt (exon 15) | wt (exons 9-20) |
| Patient #10 | F | sigmoid colon | panitumumab | yes | PR | 13 months | 4 | months | liver | liver | FFPE | wt (exon 15) | wt (exons 9-20) |
| Patient #11 | F | sigmoid-rectum | panitumumab | yes | PR | 12 months | 1 | month | paraaortic lymph | liver/paraaortic | | | |
| | | junction | | | | | | | nodes | lymph nodes | | | |

| | | Site of | | Best | | Time of biopsy | | Site of | FFPE/ | BRAF | PIK3CA |
|---|---|---|---|---|---|---|---|---|---|---|---|
| Patient ID | Gender | primary tumor | Previous chemotherapy | Response | Duration of treatment | after progression | | analysis | Frozen | mutational status | mutational status |
| Patient #13 | M | rectum | FOLFOX | PR | 4 months | 4 | months | liver | FFPE | wt (exon 15) | wt (exons 9-20) |
| Patient #14 | M | ascending | FOLFOX | PR | 5 months | 1 | month | ascending colon | FFPE | wt (exon 15) | wt (exons 9-20) |
| | | colon | | | | | | | | | |
| Patient #15 | M | sigmoid colon | FOLFOX + bevacizumab | SD | 3 months | 2 | months | liver | FFPE | wt (exon 15) | wt (exons 9-20) |
| Patient #16 | F | sigmoid colon | FOLFOX + bevacizumab; | PR | 9 months; 14 months | 1.5 | months | liver | FFPE | wt (exon 15) | wt (exons 9-20) |
| | | | bevacizumab alone | | | | | | | | |
| Patient #17 | M | sigmoid colon | FOLFOX | PR | 5 months | 2 | months | liver | FFPE | wt (exon 15) | wt (exons 9-20) |
| Patient #18 | M | descending | FOLFOX; HAIFUDR + | CR | 5 months; 2 months | 18 | months | liver | FFPE | wt (exon 15) | wt (exons 9-20) |
| | | colon | 5FU/LV | | | | | | | | |
| Patient #19 | F | ascending | capecitabine + | adjuvant | 6 months | 6 | month | liver | FFPE | wt (exon 15) | wt (exons 9-20) |
| | | colon | bevacizumab | | | | | | | | |
| Patient #21 | F | sigmoid colon | FOLFOX | PR | 3 months | 1 | month | sigmoid colon | FFPE | wt (exon 15) | wt (exons 9-20) |

FFPE: Formalin Fixed Paraffin Embedded

Table 3a and 3b, show Sanger sequencing analysis of KRAS gene.

| | Anti EGFR | Anti EGFR |
|---|---|---|
| | sensitive tumor KRAS | resistant tumor KRAS |
| | Mutational status (Sanger | Mutational status (Sanger |
| Patient ID | sequencing on tumor) | sequencing on tumor) |
| Patient #1 | wt | wt |
| Patient #2 | wt | wt |
| Patient #3 | wt | wt |
| Patient #4 | wt | wt |
| Patient #5 | wt | wt |
| Patient #6 | wt | wt |
| Patient #7 | wt | wt |
| Patient #8 | wt | Q61H |
| Patient #9 | wt | wt |
| Patient #10 | wt | wt |
| Patient #11 | wt | wt - KRAS amplified |

| | Anti EGFR | Anti EGFR |
|---|---|---|
| | sensitive tumor KRAS | resistant tumor KRAS |
| | Mutational status (454^{a} | Mutational status (454^{a} |
| Patient ID | or BEAMing^{b} on tumor) | or BEAMing^{b} on tumor) |
| Patient #2 | wt^{a} | G13D^{a} |
| Patient #7 | wt^{a} | wt^{a} |
| Patient #8 | wt^{b} | Q61H^{b} |
| Patient #9 | wt^{b} | G12D^{b} |
| Patient #10 | wt^{b} | wt^{b} |
| Patient #11 | wt^{b} | wt^{b} |

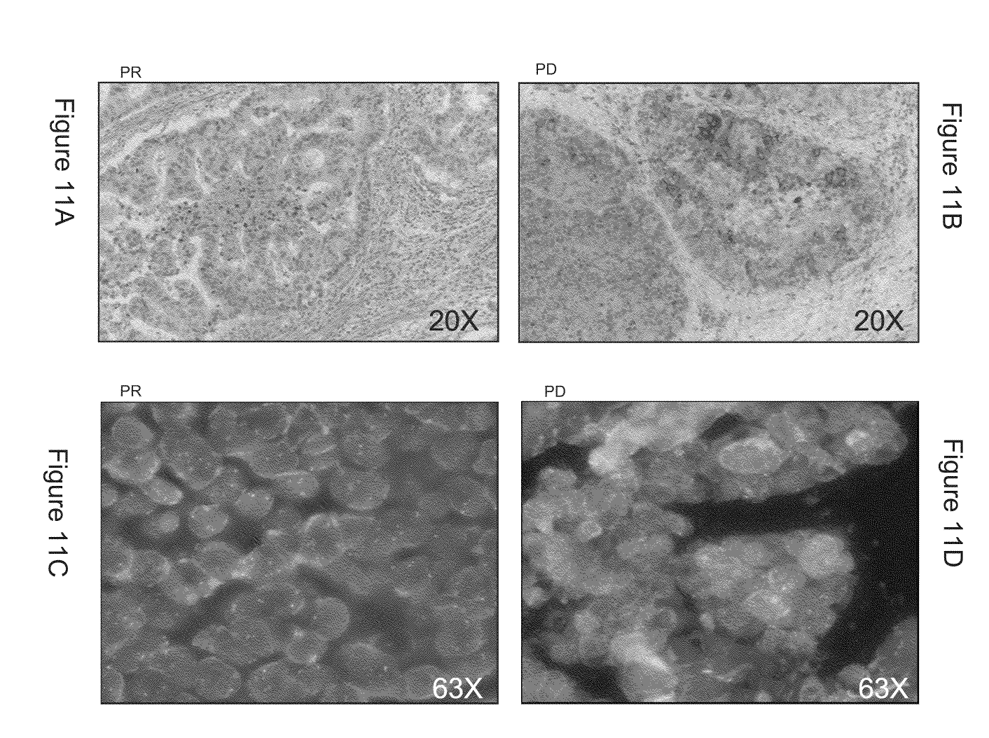

Table 3a shows Sanger sequencing analysis of KRAS gene in colorectal cancer patients tumors before and after resistance to antiEGFR therapies. Table 3b shows deep sequencing analysis of KRAS gene in colorectal cancer patients tumors before and after resistance to anti-EGFR therapies. One individual was identified (Table 3a, Patient 11) whose tumor at progression displayed KRAS amplification that was not present in a matched pre-cetuximab biopsy (FIG. 11). In a different patient, Sanger sequencing identified a KRAS Q61H mutation in a biopsy obtained following disease progression on cetuximab (Table 3a, Patient 8); whereas the remaining eight tumor samples obtained in patients with acquired resistance to anti-EGFR therapy were KRAS wild-type by this technique (Table 3a).

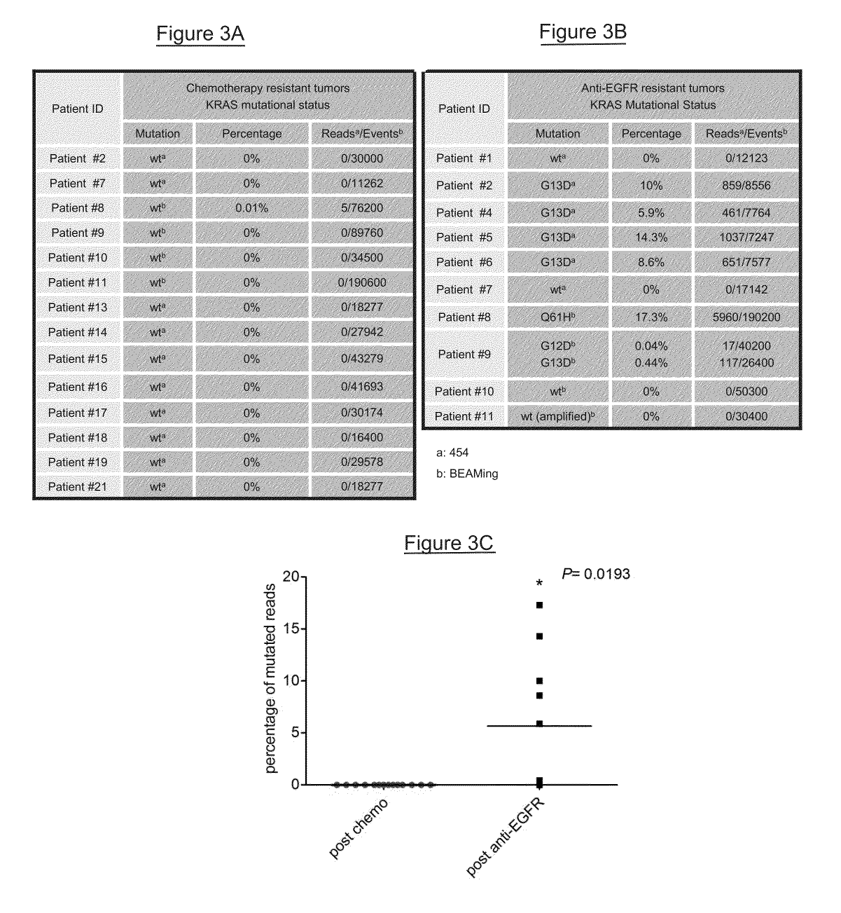

To determine whether the Sanger technology may have been underpowered to detect the presence of KRAS mutations in the biopsies obtained following cetuximab or panitumumab progression, these remaining cases were analyzed using either 454 deep sequencing or BEAMing. These techniques identified KRAS G13D mutation in four samples and the simultaneous presence of G12D and G13D mutations in one case (FIG. 3b). In the six patients for whom sufficient pre-treatment tumor samples were available for high coverage 454 sequence analysis or BEAMing, KRAS mutations were absent pre-treatment (Table 3b). Tumors from an additional eight patients treated with cytotoxic chemotherapy but not previously exposed to anti-EGFR therapies were also analyzed by 454 deep sequencing. In all eight cases (Patients #13-21), 454 analyses identified no evidence of KRAS mutation (FIG. 3a). These results indicate that treatment with anti-EGFR antibodies but not cytotoxic chemotherapy is associated with acquisition of KRAS mutations (P=0.0193) (FIG. 3c). The data support the initiation of clinical trials to define the prevalence of KRAS alterations as mechanism of acquired resistance to anti-EGFR therapies through systematic collection of biopsies.

Emergence of secondary resistance to cetuximab (disease progression) is presently established by radiological evaluation and typically occurs within 9-18 months. It was reasoned that the detection of KRAS mutant alleles in the plasma of patients treated with cetuximab or panitumumab may allow the early identification of individuals at risk for this mechanism of drug resistance prior to radiographic documentation of disease progression. Thus, BEAMing analysis of serial plasma samples from patients treated with cetuximab was performed (Tables 4a and 4b).

TABLE 4
a

| | Anti EGFR sensitive | | | Anti EGFR resistant KRAS | | |
|---|---|---|---|---|---|---|
| | KRAS mutational status in plasma | | | mutational status in plasma | | |
| Patient ID | Mutation | Percentage | Events | Mutation | Percentage | Events |
| Patient #8 | wt | 0.1% | 4/46300 | Q61H | 1.12% | 731/65400 |
| Patient #9 | wt | 0.03% | 3/11600 | G12D | 0.46\hat{ } | 89/18400 |
| | | 0% | 0/16800 | G13D | 3.3% | 427/12500 |
| Patient #10 | wt | 0% | 0/85500 | wt | 0% | 0/14200 |

b

| | | | | | | | KRAS mutational status |
|---|---|---|---|---|---|---|---|
| Patient ID | KRAS mutational status in plasma samples | | | | | | in tumor biopsy |
| Patient #8 | December 2009 | | April 2010 | | January 2011 | | February 2011 |
| | 0.01% | (Q61H) | 0.32% | (Q61H) | 1.12% | (Q61H) | 17.3% (Q61H) |
| Patient #9 | January 2011 | | March 2011 | | April 2011 | | September 2011 |
| | 0.03% | (G12D) | 0.71% | (G12D) | 0.48% | (G12D) | 0.04% (G12D) |
| | 0% | (G13D) | 1.27% | (G13D) | 3.3% | (G13D) | 0.44% (G13D) |

| | | | | |
|---|---|---|---|---|
| Patient #10 | July 2010 | April 2011 | August 2011 | November 2011 |
| | 0% | 0% | 0% | 0% |

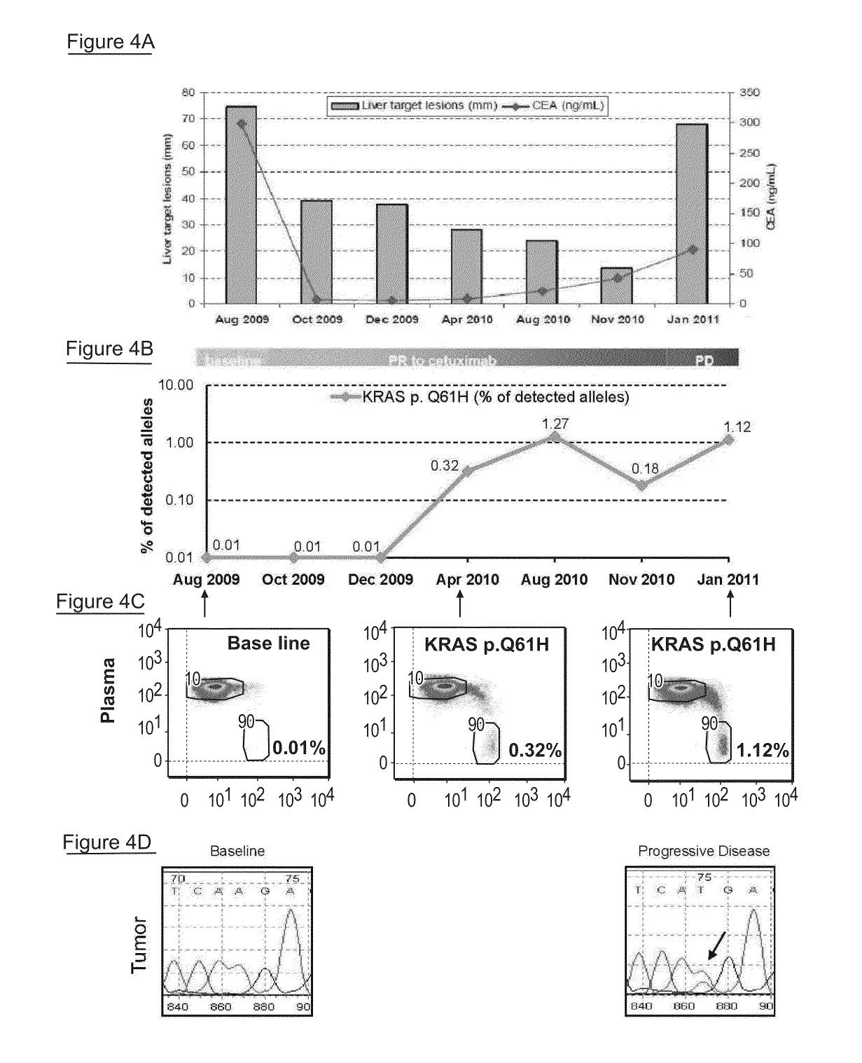

Table 4 shows BEAMing analysis of KRAS mutational status in plasma samples. Table 4a shows detection of mutated KRAS alleles in plasma of colorectal cancer patients before and after resistance to anti EGFR therapies. Table 4b shows measurements of mutant KRAS in serial plasma samples and in biopsies. The time course of plasma and bioptic sampling is indicated. This analysis confirmed that the same KRAS variants that were ultimately identified in the post-treatment (disease progression) biopsies were detectable in plasma as early as 10 months prior to the documentation of disease progression by radiological assessment (FIG. 4).

Drugs that target activated kinase pathways have profound but often temporary anti-tumor effects in subsets of patients with advanced solid tumors. In patients with advanced CRC, antibodies that bind to the extracellular domain of EGFR induce tumor regressions in 10-15% of patients when used alone and enhance the effects of cytotoxic chemotherapies when used in combination (Bardelli, A. & Siena, S. Molecular mechanisms of resistance to cetuximab and panitumumab in colorectal cancer. J Clin Oncol 28, 1254-1261 (2010); Van Cutsem, E. et al. Cetuximab and chemotherapy as initial treatment for metastatic colorectal cancer. N Engl J Med 360, 1408-1417 (2009)). The molecular basis for acquired resistance to these agents has remained obscure. The results described herein show for the first time that a substantial fraction of CRC patients who exhibit an initial response to anti-EGFR therapies have, at the time of disease progression, tumors with focal amplification or somatic mutations in KRAS which were not detectable prior to initiation of therapy.

The data indicate that drug resistance resulting from alterations in KRAS can be attributed not only the selection of pre-existent KRAS mutant and amplified clones but also to new mutations that arise as the result of ongoing mutagenesis. The percentage of KRAS mutant alleles detected in the resistant tumors ranged from 0.4 to 17% (FIG. 3). At least three (not mutually exclusive) possibilities could account for this low allele frequency. First, despite our efforts to maximize tumor content by macro dissecting each sample, the individual tumor biopsies consisted of variable proportions of tumor and intermixed KRAS wild-type stromal cells. Second, only a fraction of the tumor cells in the disease progression samples may have harbored the ‘resistance’ mutation. The latter scenario has been observed in lung cancer patients with secondary resistance to the EGFR inhibitor erlotinib where only a fraction of the tumor cells collected at the time of radiographic disease progression harbor the EGFR T790M ‘resistant’ allele (Janne, P. A. Challenges of detecting EGFR T790M in gefitinib/erlotinib-resistant tumours. Lung Cancer 60 Suppl2, 53-9, doi:50169-5002(08)70099-0 [pii] 10.1016/50169-5002(08)70099-0 (2008); Engelman, J. A. et al. Allelic dilution obscures detection of a biologically significant resistance mutation in EGFR-amplified lung cancer. J Clin Invest 116, 2695-2706 (2006); Arcila, M. E. et al. Rebiopsy of lung cancer patients with acquired resistance to EGFR inhibitors and enhanced detection of the T790M mutation using a locked nucleic acid-based assay. Clin Cancer Res 17, 1169-1180 (2011)).

Analogously, a recent study indicates that a subset of colorectal cancers found to be KRAS wild type by conventional Sanger sequencing but KRAS mutated with more sensitive techniques, do not respond to anti-EGFR treatment (Molinari, F. et al. Increased detection sensitivity for KRAS mutations enhances the prediction of anti-EGFR monoclonal antibody resistance in metastatic colorectal cancer. Clinical cancer research 17, 49014914, doi:10.1158/1078-0432.CCR-I0-3137 (2011)). These data suggest that clinical drug resistance may result from the acquisition of a drug ‘resistant’ allele in sub-population of tumor cells. Finally, it is plausible that independent cell populations harbouring different ‘resistant’ mechanisms may evolve in parallel within the same metastatic lesion. Nevertheless, functional analysis in cell models show that KRAS mutations are causally responsible for acquired resistance to cetuximab.

Finally, the KRAS mutant alleles found in the tumors of patients collected following radiographic disease progression can be detected in plasma using highly sensitive DNA analysis methods. As such tumors may be sensitive to combined treatment with a MEK inhibitor, the results suggest that blood based non-invasive monitoring of patients undergoing treatment with anti-EGFR therapies for the emergence of KRAS mutant clones could allow for the early initiation of combination therapies that may delay or prevent disease progression.

Exemplary human RAS sequences, such as two transcript variants of KRAS, are provided herein. However, all known human RAS sequences are encompassed by the invention.

Human HRAS, transcript variant 1, is encoded by the following mRNA sequence (NCBI Accession No. NM_005343 and SEQ ID NO: 1):

| | |
|---|---|
| 1 | tgccctgcgc ccgcaacccg agccgcaccc gccgcggacg gagcccatgc gcggggcgaa |
| 61 | ccgcgcgccc ccgcccccgc cccgccccgg cctcggcccc ggccctggcc ccgggggcag |
| 121 | tcgcgcctgt gaacggtggg gcaggagacc ctgtaggagg accccgggcc gcaggcccct |
| 181 | gaggagcgat gacggaatat aagctggtgg tggtgggcgc cggcggtgtg ggcaagagtg |
| 241 | cgctgaccat ccagctgatc cagaaccatt ttgtggacga atacgacccc actatagagg |
| 301 | attcctaccg gaagcaggcg gtcattgatg gggagacgtg cctgttggac atcctggata |
| 361 | ccgccggcca ggaggagtac agcgccatgc gggaccagta catgcgcacc ggggagggct |
| 421 | tcctgtgtgt gtttgccatc aacaacacca agtcttttga ggacatccac cagtacaggg |
| 461 | agcagatcaa acgggtgaag gacccggatg acgtgcccat ggtgccggtg gggaacaagt |
| 541 | gtgacctggc tgcacgcact gtggaatctc ggcaggctca ggacctcgcc cgaagctacg |
| 601 | gcacccccta catcgagacc tcggccaaga ccaggcaggg agtggaggat gccctccaca |
| 661 | cgttggtgcg tgagatccgg cagcacaagc tgcggaagct gaaccctcct gatgagagtg |
| 721 | gccccggctg catgagctgc aagtgtgtgc tctcctgacg cagcacaagc tcaggacatg |
| 781 | gaggtgccgg atgcaggaag gaggtgcaga cggaaggagg aggaaggaag gacggaagca |
| 841 | aggaaggaag gaagggccgc tggagcccag tcaccccggg accgtgggcc gaggtgactg |
| 901 | cagaccctcc cagggaggct gtgcacagac tgtcttgaac atcccaaatg ccaccggaac |
| 961 | cccagccctt agctcccctc ccaggcctct gtgggccctt gtcgggcaca gatgggatca |
| 1021 | cagtaaatta ttggatggtc ttgaaaaaaa aaaaaaaaaa a |

The amino acid sequence encoded by the mRNA sequence of SEQ ID NO: 1 is (SEQ ID NO:2):

| | | |
|---|---|---|
| 1 | MTEYKLVVVG AGGVGKSALT IQLIQNHFVD EYDPTIEDSY RKQVVIDGET CLLDILDTAG | 60 |
| 61 | QEEYSAMRDQ YMRTGEGFLC VFAINNTKSF EDIHQYREQI KRVKDSDDVP MVLVGNKCDL | 120 |
| 121 | AARTVESRQA QDLARSYGIP YIETSAKTRQ GVEDAFYTLV REIRQHKLRK LNPPDESGPG | 180 |
| 101 | CMSCKCVLS | |

Human HRAS, transcript variant 2, is encoded by the following mRNA sequence (NCBI Accession No. NM_176795 and SEQ ID NO: 3):

| | |
|---|---|
| 1 | tgccctgcgc ccgcaacccg agccgcaccc gccgcggacg gagcccatgc gcggggcgaa |
| 61 | ccgcgcgccc ccgcccccgc cccgccccgg cctaggcccc ggccctggcc ccgggggcag |
| 121 | tcgcgcctgt gaacggtggg gcaggagacc ctgtaggagg accccgggcc gcaggcccct |
| 161 | gaggagcgat gacggaatat aagctggtgg tggtgggcgc cggcggtgtg ggcaagagtg |
| 241 | cgctgaccat ccagctgatc cagaaccatt ttgtggacga atacgacccc actatagagg |
| 301 | attcctaccg gaagcaggtg gtcattgatg gggagacgtg cctgttggac atcctggata |
| 361 | ccgccggcca ggaggagtac agcgccatgc gggaccagta catgcgcacc ggggagggct |
| 421 | tcctgtgtgt gtttgccatc aacaacacca agtcttttga ggacatccac cagtacaggg |
| 481 | agcagatcaa acgggtgaag gactcggatg acgtgcccat ggtgctggtg gggaacaagt |
| 541 | gtgacctggc tgcacgcact gtggaatctc ggcaggctca ggacctcgcc cgaagctacg |
| 601 | gcatccccta catcgagacc tcggccaaga cccggcaggg cagccgctct ggctctagct |
| 661 | ccagctccgg gaccctctgg gaccccccgg gacccatgtg acccagcggc ccctcgcgct |
| 721 | ggagtggagg atgccttcta cacgttggtg cgtgagatcc ggcagcacaa gctgaggaag |
| 781 | ctgaaccctc ctgatgagag tggccccggc tgcatgagct gcaagtgtgt gctctcctga |
| 841 | ctgaaccctc gctcaggaca tggaggtgcc ggatgcagga aggaggtgca gacggaagga |
| 901 | ggaggaagga aggacggaag caaggaagga aggaagggct gctggagccc agtcaccccg |
| 961 | ggaccgtggg ccgaggtgac tgcagaccct cccagggagg ctgtgcacag actgtcttga |
| 1021 | acatcccaaa tgccaccgga accccagccc ttagctcccc tcccaggcct ctgtgggccc |
| 1081 | ttgtcgggca cagatgggat cacagtaaat tattggatgg tcttgaaaaa aaaaaaaaaa |
| 1141 | aaa |

The amino acid sequence encoded by the mRNA sequence of SEQ ID NO: 3 is (SEQ ID NO:4):

| | | |
|---|---|---|
| 1 | MTEYKLVVVG AGGVGKSALT IQLIQNHFVD EYDPTIEDSY RKQVVIDGET CLLDILDTAG | 60 |
| 61 | QEEYSAMRDQ YMRTGEGFLC VFAINNTKSF EDIHQYREQI KRVKDSDDVP MVLVGNKCDL | 120 |
| 121 | AARTVESRQA QDLARSYGIP YIETSAKTRQ GSRSGSSSSS GILWDPPGPM | |

Having now generally provided the disclosure, the same will be more readily understood through reference to the following examples which are provided by way of illustration, and are not intended to be limiting of the disclosure, unless specified.

### EXAMPLES

### Example 1

### Methods and Materials

DiFi and Lim1215 were exposed to different doses of cetuximab as described in figure S2 to obtain the resistant variants. Cell viability was assessed by ATP content. Cells were seeded in 100 μl medium in 96-well plastic culture plates. The experimental procedures for knock in of cancer mutations, the vectors. AA V production, cell infection and screening for recombinants have already been described elsewhere.

Tumor specimens were obtained through protocols approved by the Institutional Review Board of Memorial Sloan-Kettering Cancer Center (protocol 10-029) and Ospedale Niguarda Ca' Granda, Milano, Italy (protocols 1014/09 and 194/2010). Details about the clinical characteristics of patients are provided in Table 2. Identification of cancer mutations in the KRAS, HRAS, NRAS, BRAF, PIK3CA and EGFR genes was performed with different sequencing platforms (Sanger, 454 pyrosequencing and Mass Spectrometry) as described herein.

For immunoblot analysis, total cellular proteins were extracted by solubilizing the cells in boiling SDS buffer. Western blot detection was done by enhanced chemiluminescence. The analysis of KRAS activation was performed by immunoprecipitation assay with GST-Raf1-RBD. Real time PCR was performed using an ABI PRISM® 7900HT apparatus (Applied Biosystems). KRAS protein expression was evaluated by immunohistochemistry performed on 3 μm thick tissue sections using a specific KRAS (F234) antibody (SC-30, mouse monoclonal IgG₂ₐ Santa Cruz Biotechnology). BEAMing was performed essentially as described previously (De Roock, W. et al. Effects of KRAS, BRAF, NRAS, and PIK3CA mutations on the efficacy of cetuximab plus chemotherapy in chemotherapy-refractory metastatic colorectal cancer: a retrospective consortium analysis. Lancet Oncol. 11, 753-762 (2010)), deviation from the original protocol are outlined hereinbelow. FISH experiments were conducted according with the histology FISH accessory kit (Dako, Glostrup, Danmark). Data are presented as the mean±SD and n=3. Statistical significance was determined by paired Student's t test. P<0.05 was considered statistically significant.

### Example 2

### Cell Culture and Generation of Resistant Cells

DiFi cells were cultured in F12 medium (Invitrogen) supplemented with 5% FBS and Lim1215 cells were cultured in RPMI-1640 medium (Invitrogen) supplemented with 5% FBS and Insulin (1 μg/ml). DiFi parental cells were plated in 100 mm Petri dishes with 2.5% FBS and exposed to a constant dose of cetuximab (350 nM), for one year in order to obtain the resistant counterpart DiFi R1. The DiFi R2 derivative was obtained by increasing cetuximab dosage stepwise starting from 3.5 nM, to 35 nM and finally to 350 nM during the course of one year. The same protocols were applied to Lim1215 with variations regarding cetuximab concentrations: for Lim R1 cetuximab was used at 1400 nM and for Lim R2 drug concentration started from 350 nM, to 700 nM and finally 1400 nM. For Lim1215 both protocols required at least 3 months' drug treatment. The Lim1215 parental cell line had been described previously (Whitehead, R. H., Macrae, F. A., St John, D. J. & Ma, J. A colon cancer cell line (LIM1215) derived from a patient with inherited nonpolyposis colorectal cancer. J Natl Cancer 174, 759-765 (1985)) and was obtained from Prof Robert Whitehead. Vanderbilt University, Nashville, with permission from the Ludwig Institute for Cancer Research, Zurich, Switzerland. The genetic identity of the cell lines used in this study was confirmed by STR profiling (Cell ID, Promega)

### Example 3

### Drug Viability Assays

Cetuximab was obtained from Pharmacy at Niguarda Ca' Granda Hospital, Milan, Italy. AZD6244 and GSK1059615 were purchased from Sequoia Chemicals (Pangbourne, UK) and Selleck Chemicals (Houston, USA), respectively. Cell lines were seeded in 100 μi medium at appropriate density (5×10⁴, 1.5×10⁴ for DiFi and Lim1215 cells, respectively) in 96-well plastic culture plates. After serial dilutions, drugs in serum free medium were added to cells and medium-only containing wells were added as controls. Plates were incubated at 37° C. in 5% CO₂ for 72-168 h, after which cell viability was assessed by ATP content using the CellTiter-Glo® Luminescent Assay (Promega Madison, Wis., USA).

### Example 4

### Mutational Analysis

RAS genotyping was performed using the iPLEX assay (Sequenom, Inc.), which is based on a single-base primer extension assay. Briefly, multiplexed PCR and extension primers are designed for a panel of known mutations. After PCR and extension reactions, the resulting extension products are analyzed using a matrix-assisted laser desorption/ionization-time-of-flight (MALDI-TOF) mass spectrometer. For 454 picotiter plate Pyrosequencing (Roche Inc.), PCR products were generated using primers designed to span exons 2, 3 and 4 in KRAS and adapted with 5′ overhangs to facilitate emulsion polymerase chain reaction (emPCR) and sequencing. After amplification by emPCR, beads containing DNA were isolated. 34,000 beads were sequenced in both directions, yielding 1000-5000 sequencing reads on average per sample (˜1000 reads per amplicon per sample) using GSFLX. To detect variants in 454 sequencing data, reads were mapped with the BWA aligner using the bwasw mode for aligning long reads. The generated SAM file was then run through the Picard MarkDuplicate program to remove duplicated reads (reads with the same initial starting point). The file was then processed with the GATK BaseQ recalibrator. Finally, pileups were generated using Samtools and called variants using VarScan. For Sanger Sequencing all samples were subjected to automated sequencing by ABI PRISM 3730 (Applied Biosystems, Foster City, Calif., USA). All mutations were confirmed twice, starting from independent PCR reactions.

All primer sequences are available upon request to the inventors. Exome sequencing was carried out by exome capture using the SeqCap EZ Human Exome Library v1.0 (Nimblegen Inc.) and subsequent pyrosequencing of the captured fragments by means of 454Flx sequencer (Roche Inc.), according to manufacturer's protocols. A total of 1.2 million reads were sequenced for an average exome depth of 4×. The reads were mapped using the manufacturer's mapping tools and the reads' depth used as an estimator of the copy number value in the two DiFi parental and DiFi resistant samples. Average reads' depths were calculated within overlapping 100,000 bp wide windows for FIG. 1 b, while average reads' depths were calculated for exons and genes and respectively plotted as dots and segments in FIGS. 7a and 7b.

### Example 5

### Tissue Procurement

Tumor specimens were obtained through protocols approved by the Institutional Review Board of Memorial Sloan-Kettering Cancer Center (protocol 10-029) and Ospedale Niguarda Ca' Granda, Milano, Italy (protocols 1014/09 and 194/2010). All tumor specimens were formalin fixed paraffin embedded (FFPE). All patients provided informed consent and samples were procured and the study was conducted under the approval of the Review Boards and Ethical Committees of the Institutions. Details about the clinical characteristic of the patients are provided in Table 2.

### Example 6

### BEAMing Procedure

DNA was extracted from plasma using the QIAamp Circulating Nucleic Acid Kit (QIAGEN) according to manufacturer's instructions. BEAMing was performed as described previously. The first amplification was performed in 50-μl PCR reaction, containing DNA isolated from 1 ml of plasma, IX Phusion high-fidelity buffer, 1.5 U of Hotstart Phusion polymerase (NEB, BioLabs), 0.5 μl of each primer with tag sequence, 0.2 mM of each deoxynucleoside triphosphate, and 0.5 mmol/L MgCl₂. Amplification was carried out using the following cycling conditions: 98° C. for 45 sec; 2 cycles of 98° C. for 10 sec, 67° C. for 10 sec, 72° C. for 10 sec; 2 cycles of 98° C. for 10 sec, 64° C. for 10 sec, 72° C. for 10 sec; 2 cycles of 98° C. for 10 sec, 61° C. for 10 sec, 72° C. for 10 sec; 31 cycles of 98° C. for 10 sec, 58° C. for 10 sec, 72° C. for 10 sec.

PCR products were diluted, and quantified using the PicoGreen double-stranded DNA assay (Invitrogen, Carlsbad, Calif.). A clonal bead population is generated performing an emulsion PCR (emPCR). 150 μl PCR mixture was prepared containing 18 pg template DNA, 40 U of Platinum Taq DNA polymerase (Invitrogen), IX Platinum buffer, 0.2 mM dNTPs, 5 mM MgCl₂, 0.05 μl Tag1 (5′-tcccgcgaaattaatacgac, SEQ ID NO:5), 8 μM Tag2 (5′-gctggagctctgcagcta, SEQ ID NO:6) and 6×10⁷ magnetic streptavidin beads (MyOne, Invitrogen) coated with Tag1 oligonucleotide (dual biotin Tspacer18-tcccgcgaaattaatacgac, SEQ ID NO:5). The 150 μl PCR reactions were distributed into the wells of a 96-well PCR plate together with 70 μl of the Emusifire oil. The water-in-oil emulsion was obtained by pipetting. The PCR cycling conditions were: 94° C. for 2 min, 50 cycles of 94° C. for 10 sec, 58° C. for 15 sec, 70° C. for 15 sec. All primer sequences are available upon request to the inventors.

### Example 7

### Immunoblot Analysis

Prior to biochemical analysis, all cells were grown in their specific media supplemented with 5% FBS. Total cellular proteins were extracted by solubilizing the cells in boiling SDS buffer (50 mM Tris-HCl, pH 7.5, 150 mM NaCl, and 1% SDS). Western blot detection was done by enhanced chemiluminescence (GE Healthcare). The following antibodies were used for western blotting (all from Cell Signaling Technology, except where indicated): anti-phosphoAKT S473; anti-phospho-AKT T308; anti-AKT: anti-phospho-p44/42 ERK (thr202/tyr204); anti-p44/42 ERK; anti-P-MEK1/2 (Ser217/221), anti-MEK1/2; anti-KRAS (Santa Cruz); antiEGFR (clone 13G8, Enzo Life Sciences); anti-phospho EGFR (tyr1068); anti-actin and antivinculin (Sigma-Aldrich, St. Louis, Mo.).

### Example 8

### KRAS Activation Assay (RAS-GTP)

The analysis of KRAS activation was performed by an immunoprecipitation assay with GST-Raf1-RBD (ras binding domain), as previously described (Di Nicolantonio, F. et al. Replacement of normal with mutant alleles in the genome of normal human cells unveils mutation-specific drug responses. Proc. Natl. Acad. Sci. USA. 105, 20864-20869, doi:10.1073/pnas.0808757105 (2008)). The KRAS protein was detected with Anti-KRAS (F234) mAb (Santa Cruz, Santa Cruz, Calif.).

### Example 9

### Gene Copy Number Analysis (gPCR and NGSeq)

Parental and resistant cell lines were trypsinized, washed with PBS and centrifuged; pellets were lysed and DNA was extracted using Wizard SV Genomic kit (Promega) according to the manufacturer's directions. Real time PCR was performed with 150 ng of DNA per single reaction using GoTaq QPCR Master Mix (Promega) and determined by real time PCR using an ABI PRISM® 7900HT apparatus (Applied Biosytems).

All primer sequences are available upon request to the inventors. Exome sequencing was carried out by exome capture using the SeqCap EZ Human Exome Library v1.0 (Nimblegen Inc.) and subsequent pyrosequencing of the captured fragments by means of 454Flx sequencer (Roche Inc.), according to manufacturer's protocols. A total of 1.2 Million reads were sequenced for an average exome depth of 4×. The reads were mapped using the manufacturer's mapping tools and the reads' depth was determined and used as an estimator of the copy number value in the two DiFi parental and DiFi resistant samples. Average reads' depths within overlapping 100,000 bp wide windows were calculated and plotted in FIG. 1c; average reads' depths within exons and genes were calculated and respectively plotted as dots and segments in FIGS. 8a and 8b.

### Example 10

### Immunohistochemistry Assay

KRAS protein expression was evaluated by immunohistochemistry performed on 3 p, m thick tissue sections using a specific KRAS (F234) antibody (SC-30, mouse monoclonal IgG2a Santa Cruz Biotechnology; dilution 1:100) and the automated system BenchMark Ultra (Ventana Medical System, Inc., Roche) according to the manufacturer's instructions, with minimum modifications. KRAS protein expression was detected at cytoplasmatic and membrane level. Samples were considered positive when the expression of protein was present in at least 10% of cells. Healthy tissue, i.e, normal colon mucosa, was used as internal negative control; slide with DiFi R2 cell line was used as external positive control. Images were captured with the AxiovisionLe software (Zeiss, Gottingen, Germany) using a Axio Zeiss Imager 2 microscope (Zeiss, Gottingen, Germany).

### Example 11

### Fluorescent In Situ Hybridisation (FISH) Analysis

All analyses were performed on 3 m thick sections of formalin-fixed paraffin-embedded tumour tissue, provided by the department of anatomy pathology of Niguarda Hospital, and on metaphase chromosomes and interphase nuclei, obtained from DiFi cell line culture following standard procedures. Tissue sections for FISH experiment were prepared according to the manufacturer's instructions of Histology FISH Accessory kit (Dako, Glostrup, Danmark). For both types of samples the last steps before hybridization were: dehydration in ethanol series (70%, 90%, 100%), 3 washes (5′ each) and air drying.

Dual colour FISH analysis was performed using a 10 μl mix-probe made up by 1 μl CEP12 alpha satellite probe (12p11-q11) labeled in SpectrumOrange (Vysis, Downers Grove, Ill. USA), 1 μl BAC (Bacterial Artificial Chromosome) genomic probe RP11-707G18 (12p12.1) spanning an approximately 176 kb region encompassing the KRAS gene, labeled in SpectrumGreen (Bluegnome; Smith, G. et al. Activating K-Ras mutations outwith ‘hotspot’ codons in sporadic colorectal tumours—implications for personalised cancer medicine. Br. J Cancer 102, 693-703, doi:10.1038/sj.bjc.6605534 (2010)) and 8 μl LSI-WCP hybridisation buffer (Vysis, Downers Grove, Ill. USA) for each slide. Probes and target DNA of specimens were co-denatured in HYBRite System (Dako Glostrup, Danmark) for 5 min at 75° C. and then hybridized overnight at 37° C. Slides were washed with post-hybridisation buffer (Dako Glostrup, Danmark) at 73° C. for 2 min and counterstained with 4,6-diamino-2phenylindole (DAPI II: Vysis, Downers Grove, Ill. USA). Fluorescent in situ hybridisation signals were evaluated with a Zeiss Axioscope Imager. ZI (Zeiss, Gottingen, Germany) equipped with single and triple band pass filters. Images for documentation were captured with CCD camera and processed using the MetaSystems Isis software. Samples with a ratio greater than 3 between KRAS gene and chromosome 12 centromere signals, in at least 10% of 100 cells analysed in 10 different fields, were scored as positive for KRAS gene amplification. Healthy tissue, i.e, normal colon mucosa, was used as internal negative control.

### Example 12

### Plasmids and Viral Vectors

All experimental procedures for targeting vector construction, AAV production, cell infection and screening for recombinants have already been described elsewhere (Di Nicolantonio, F. et al. Replacement of normal with mutant alleles in the genome of normal human cells unveils mutation-specific drug responses. Proc. Natl. Acad. Sci. USA. 105, 20864-20869, doi: 10.1073/pnas. 0808757105 (2008)).

### Example 13

### Statistical Analysis

Data are presented as the mean±SD and n=3. Statistical significance was determined by paired Student's t test or two-tailed unpaired Mann-Whitney test (FIG. 3c). P<0.05 was considered statistically significant.

All references cited herein, including patents, patent applications, and publications, are hereby incorporated by reference in their entireties, whether previously specifically incorporated or not.

Having now fully described the inventive subject matter, it will be appreciated by those skilled in the art that the same can be performed within a wide range of equivalent parameters, concentrations, and conditions without departing from the spirit and scope of the disclosure and without undue experimentation.

While this disclosure has been described in connection with specific embodiments thereof, it will be understood that it is capable of further modifications. This application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth.

What is claimed is:

1. A method of predicting if a subject being treated for colorectal cancer with anti-EGFR therapy will develop drug resistance, the method comprising,
obtaining a biological sample from said subject, and assaying said sample for an alteration in KRAS expression,
wherein if an alteration in KRAS expression is detected, the subject is more likely to develop drug resistance to said anti-EGFR therapy.

2. The method of claim 1, wherein said alteration in KRAS expression is the presence of a KRAS mutant.

3. The method of claim 2, wherein said KRAS mutant is selected from a G13D mutation, a G12R mutation, a Q61H mutation, or an A146T mutation in the human KRAS amino acid sequence of SEQ ID NO: 2 or 4.

4. The method of claim 1, wherein said alteration in KRAS expression is determined by comparing the expression of KRAS in said sample to the expression of KRAS in a control, non-cancerous biological sample.

5. The method of claim 4, wherein said alteration in said KRAS expression is an increase in nucleic acid or protein expression, or an increase in KRAS functional activity, when compared to said control, non-cancerous biological sample.

6. The method of claim 4, wherein said alteration in said KRAS expression is a decrease in nucleic acid or protein expression, or a decrease in KRAS functional activity, when compared to said control, non-cancerous biological sample.

7. The method of claim 1, wherein the anti-EGFR therapy is treatment with cetuximab or panitumumab.

8. The method of claim 1, wherein the biological sample is blood, plasma, serum, urine, tissue, cells or a biopsy.

9. A method of preventing, reducing or delaying the onset of drug resistance to anti-EGFR therapy, the method comprising,
administering, to a subject having an alteration in KRAS expression, a MEK inhibitor in combination with said anti-EGFR therapy.

10. The method of claim 9, wherein said alteration in KRAS expression is the presence of a KRAS mutant.

11. The method of claim 10, wherein said KRAS mutant is a G13D mutation, a G12R mutation, a Q61H mutation, or an A146T mutation in the human KRAS amino acid sequence of SEQ ID NO: 2 or 4.

12. The method of claim 9, wherein said alteration in KRAS expression is determined by comparing the expression of KRAS in a biological sample from a subject being treated with anti [symbol] EGFR therapy to the expression of KRAS in a control, non-cancerous biological sample.

15. The method of claim 12, wherein said alteration in said KRAS expression is an increase in nucleic acid or protein expression, or an increase in KRAS functional activity, when compared to said control, non-cancerous biological sample.

16. The method of claim 12, wherein said alteration in said KRAS expression is a decrease in nucleic acid or protein expression, or a decrease in KRAS functional activity, when compared to said control, non-cancerous biological sample.

17. The method of claim 9, wherein the anti-EGFR therapy is treatment with cetuximab or panitumumab; or
wherein the biological sample is blood, plasma, serum, urine, tissue, cells or a biopsy; or
wherein the subject is diagnosed with colorectal cancer; or
wherein the MEK inhibitor is XL 518, CI-I040, PD035901, GSK1120212 or selumetinib.

18. A method of detecting a KRAS mutation, the method comprising assaying a biological sample from a human subject for a KRAS mutation selected from G13D, G12R, Q61H, and A146T in the human KRAS amino acid sequence of SEQ ID NO: 2 or 4.

19. The method of claim 18, wherein said assaying comprises sequencing a DNA or cDNA oligonucleotide to detect the KRAS mutation.

20. The method of claim 18, wherein said assaying comprises amplification, of a DNA or cDNA oligonucleotide comprising a sequence containing the KRAS mutation, with at least one primer complementary to all or part of said oligonucleotide.


SEQUENCE LISTING (6 sequences)

SEQ ID NO: 1 - DNA, 1061 bp, Homo sapiens
tgccctgcgcccgcaacccgagccgcacccgccgcggacggagcccatgcgcggggcgaaccgcgcgcccccgcccccgccccgccccggcctcggccccggccctggccccgggggcagtcgcgcctgtgaacggtggggcaggagaccctgtaggaggaccccgggccgcaggcccctgaggagcgatgacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggagtggaggatgccttctacacgttggtgcgtgagatccggcagcacaagctgcggaagctgaaccctcctgatgagagtggccccggctgcatgagctgcaagtgtgtgctctcctgacgcagcacaagctcaggacatggaggtgccggatgcaggaaggaggtgcagacggaaggaggaggaaggaaggacggaagcaaggaaggaaggaagggctgctggagcccagtcaccccgggaccgtgggccgaggtgactgcagaccctcccagggaggctgtgcacagactgtcttgaacatcccaaatgccaccggaaccccagcccttagctcccctcccaggcctctgtgggcccttgtcgggcacagatgggatcacagtaaattattggatggtcttgaaaaaaaaaaaaaaaaaa

SEQ ID NO: 2 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 3 - DNA, 1143 bp, Homo sapiens
tgccctgcgcccgcaacccgagccgcacccgccgcggacggagcccatgcgcggggcgaaccgcgcgcccccgcccccgccccgccccggcctcggccccggccctggccccgggggcagtcgcgcctgtgaacggtggggcaggagaccctgtaggaggaccccgggccgcaggcccctgaggagcgatgacggaatataagctggtggtggtgggcgccggcggtgtgggcaagagtgcgctgaccatccagctgatccagaaccattttgtggacgaatacgaccccactatagaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcagggcagccgctctggctctagctccagctccgggaccctctgggaccccccgggacccatgtgacccagcggcccctcgcgctggagtggaggatgccttctacacgttggtgcgtgagatccggcagcacaagctgcggaagctgaaccctcctgatgagagtggccccggctgcatgagctgcaagtgtgtgctctcctgacgcagcacaagctcaggacatggaggtgccggatgcaggaaggaggtgcagacggaaggaggaggaaggaaggacggaagcaaggaaggaaggaagggctgctggagcccagtcaccccgggaccgtgggccgaggtgactgcagaccctcccagggaggctgtgcacagactgtcttgaacatcccaaatgccaccggaaccccagcccttagctcccctcccaggcctctgtgggcccttgtcgggcacagatgggatcacagtaaattattggatggtcttgaaaaaaaaaaaaaaaaaa

SEQ ID NO: 4 - protein, 170 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGSRSGSSSSSGTLWDPPGPM

SEQ ID NO: 5 - DNA, 20 bp, Artificial Sequence
  Chemically synthesized oligonucleotide
tcccgcgaaattaatacgac

SEQ ID NO: 6 - DNA, 18 bp, Artificial Sequence
  Chemically synthesized oligonucleotide
gctggagctctgcagcta